(19) United States
(12) Patent Application Publication
(10) Pub. No.: US 2006/0246025 A1
(43) Pub. Date: Nov. 2, 2006

(54) Natural fast-drying hair fixative compositions

(76) Inventors: Michael Thomas Hayes, Centerville, MN (US); Alex Vainshelboim, Maple Grove, MN (US); Kenneth S. Momoh, St. Paul, MN (US); Patricia L. Peterson, Stillwater, MN (US); Peter Matravers, Minnetonka, MN (US); Anita Grahn, Blaine, MN (US)

Correspondence Address: THE ESTEE LAUDER COS, INC, 125 PINELAWN ROAD, MELVILLE, NY, 11747, US

(21) Appl. No.: 11/117,769

(22) Filed: Apr. 28, 2005

(51) Int. Cl. A61K 8/73

(52) U.S. Cl. 424/70.7; 424/70.13

(57) ABSTRACT

The present invention is a combination of a linear polysaccharide such as pullulan and a monohydric alcohol in certain amounts. The monohydric alcohol is present in drying time enhancing effective amounts such that drying times decrease with an increase in alcohol. Pullulan is used as the hair fixative agent, and a wetting agent can be added to the hair fixative compositions of the present invention.

### FIELD OF THE INVENTION

The present invention relates to naturally derived linear polysaccharides and their use in natural hair care products. In particular, the invention relates to fast-drying hair styling compositions containing pullulan as the hair fixative agent for the keratinous surface.

### BACKGROUND OF THE INVENTION

After styling hair into a particular hair fashion, the ability to hold the hair style is of next importance. In order for a hair spray or hair fixative composition to hold the hair, an effective hair spray formulation and film forming resin contained therein must meet certain characteristics. Specifically, the film forming resins should remain non-tacky in a humid environment but be easily removed upon use of soap solutions such as shampoos. The film should have high hair cohesivity and possess sufficient strength and elasticity to avoid dusting or flaking when the hair is subjected to combing or brushing stresses. Further, the resin should remain clear, transparent and glossy on aging. The film forming resin should have a viscosity range which permits spraying without causing the nozzle to clog, and yet not be too thin as a liquid. The resin should also show little or no tendency to interact with perfumes or other optional components typically utilized in a hair spray formulation.

The early hair fixative compositions contained shellac, a natural resinous material derived from the secretion of the insect Laccifer lacca that formed a water-insoluble film on the hair. Thus, the shellac had to be solubilized for use in an aqueous system. Consequently, the shellac did not wash out of the hair with relative ease as desired for a hair fixative. To remedy this issue, synthetic polymers were developed and incorporated into hair fixative compositions. Specifically, polyvinylpyrrolidone (PVP) was introduced. PVP is a water-soluble material that adheres well to hair and produces a clear, flexible film on the hair surface. Much like shellac, PVP as a pure homopolymer had its drawbacks as a fixative agent. Its hygroscopicity caused it to absorb moisture in the air and become sticky on the hair. This, therefore, spurred on a second generation of PVP copolymers such as, for example, PVP vinyl acetate (PVP-VA). The development of synthetic polymers continues; however, efforts in this regard do not address the growing demand for the use of natural ingredients in personal care products, including hair care products. Additional concerns pertain to a failure to achieve a smooth uniform film on the hair shaft, poor curl retentivity due to the lumpy islands formed instead of a smooth film, a dusting-off phenomenon of resin particles, and static charges experienced with these types of conventional hair fixative agents.

In addition to improving the qualities of film forming resins, there has been an increasing desire for natural ingredients and natural products. One natural film former that has been proposed as a film forming agent is xanthan gum as disclosed in US Application Publication No. 2003/0108505 A1. Xanthan gum, a polysaccharide gum derived from the bacterium Xanthomonas and having a main chain comprising β-(1,4) D-glucose units, is alleged to be advantageous as a fixative in hair compositions because it can be used with other hair fixatives and provides rheology modifying properties. Other advantageous properties noted in association with xanthan gum are stiffness, gloss, dry comb, wet comb, non-flakiness, anti-static, feel and high humidity curl retention. Some of these properties however, are somewhat contradictory. A stiff polymer will be generally less flexible; less flexibility gives rise to increased flakiness. There are certain known drawbacks associated with the use of xanthan—lack of transparency, high viscosity, holding performance and brittleness. Higher viscosities are also associated with difficulties in spraying. Some of these drawbacks are the result of its highly branched structure or the presence of side chains. Examples of other more common natural polymers are chitosan (derived from the polysaccharide chitin), alginates, gelatin, pectins, and polysaccharides such as cellulose derivatives, starch or guar gum are known but contain highly branched structures or side chains that can greatly restrict the packing characteristics of the polymer. Another example of a natural fixative agent is pullulan. Unlike xanthan, (the disclosed polymer in Patent 2003/0108505), pullulan is a linear high molecular weight homopolysaccharide of glucose having a unique linkage pattern. While not wishing to be bound to a particular theory, its linear structure is believed to be responsible for many of its favorable properties. Pullulan and its glucose units are entirely different in molecular structure from chitosan, starches, cellulose, amylose, celluloses, and similar polysaccharides or derivatives which are also glucose derivatives. The difference in molecular structure between pullulan and other polymers is exhibited by the distinct properties between pullulan and the other glucose-unit containing polymers.

Produced as a water-soluble, extracellular polysaccharide by certain strains of a polymorphic fungus such as, for example, Aureobasidium pullulans or Dematium pullulans, pullulan has become well known for its adhesive properties and is used in a variety of products ranging from breath mint strips to compression moldings. Pullulan is known to be an excellent film former and can be processed into a strong film which can be transparent. Its use in a hair product such as a setting lotion and a hair lacquer is disclosed in U.S. Pat. No. 3,972,997 as a tacky ingredient with transparent film forming ability. The film is made by casting and drying a pullulan solution. The pullulan film is soluble in water, including cold water, and is glossy, transparent, tough, and heat sealable. The adhesiveness of pullulan is developed by adding water.

Achieving the characteristics of a film fixative agent in a hair composition can present a formidable task. The introduction of water into the hair fixative composition can adversely affect the performance of the composition. Water also has the disadvantage of increasing the drying time of the hair styling composition after it is applied to the hair when compared to hair styling compositions containing higher levels of VOC. Water can also adversely affect the properties of the hair fixative resin. For example, if the hair fixative resin is water soluble, water can cause the composition to have high viscosities (greater than 15 centipoise) leading to decreased sprayability of the hair styling composition and clogging of the spray nozzle. Therefore, the object of the present invention is to incorporate pullulan as a natural aqueous hair fixative agent in hair care products that meet the desired traits of applicability, hold, adhesion, transparency, and washability, and overcomes challenges in formulating a fast-drying hair fixative composition.

### SUMMARY OF THE INVENTION

The present invention relates to a hair fixative composition comprising a linear polysaccharide, pullulan, having a molecular weight of 5,000 to 5,000,000 as a fixative agent. The pullulan is a linear and unbranched polysaccharide, i.e., the polysaccharide is free of side chains. The fixative agent is present in the hair composition in an amount of about 0.5 to 15 percent by weight of the composition. The hair composition has a decreased drying time. Pullulan is present in an hydroalcoholic solvent system comprising amounts of monohydric alcohol effective in decreasing the drying time of the hair fixative composition. Specifically, the monohydric alcohol is present in amounts of about 15 to 35 percent by weight of the composition. The linear polysaccharide, pullulan, provides a strong yet thin film on the hair which acts as the hair fixative agent, free from many conventional performance drawbacks. In addition, the hair compositions of the present invention containing pullulan as the hair fixative have the benefit of being fast-drying. This is a surprising and unexpected result observed with the increased amounts of ethanol in the hydroalcoholic system.

For example, the water/alcohol combinations of the present invention have the effect of decreasing the drying time of the hair fixative composition as well as sprayability issues as compared with other hair fixative compositions. Because of the intrinsic chemical and mechanical properties of pullulan, no additional plasticizer is needed. Moreover, pullulan films, due to their limited permeability to oxygen and other gases, offer enhanced protection to the hair against environmental factors such as cigarette smoke, carbon monoxide, oxygen free radicals, and other airborne sources of pollution. The present invention also relates to methods of preparing the compositions containing pullulan, and to methods of setting the hair and decreasing the drying time of the hair fixative compositions.

### DESCRIPTION OF THE DRAWINGS

FIG. 1 is a line chart depicting the drying time of compositions containing various percentages of ethanol in combination with 1 percent pullulan in terms of percent weight loss; specifically, 0, 5, 10, 25 and 30 percent ethanol.

FIG. 2 is a line chart depicting the drying time of compositions containing various percentages of ethanol in combination with 5 percent pullulan in terms of percent weight loss; specifically, 0, 5, 10, 25 and 30 percent ethanol.

FIG. 3 is a line chart depicting the drying time of compositions containing various percentages of ethanol in combination with 10 percent pullulan in terms of percent weight loss; specifically, 0, 5, 10, 25 and 30 percent ethanol.

### DETAILED DESCRIPTION OF THE INVENTION

The presence of a linear polysaccharide in the hair compositions of the present invention provides a strong yet thin film capable of acting as a fixative agent. In addition to forming a strong film, the linear polysaccharide, pullulan, has high adhesion strength and good water solubility. The hydroxyl groups in the repeat units of pullulan provide for excellent water solubility, and therefore, it is not necessary to form salts to achieve solubilization. Because this polymer is completely soluble in water, the film formed on the surface of the hair shaft is easily removed with soap, and especially with shampoo. The resultant film on the hair shaft also does not flake or dust-off when combing force is applied. In addition, the hair fixative compositions of the present invention do not contain heavy metals or cross-linking ingredients, catalysts or complexes. As the sole hair fixative agent, pullulan forms a strong, yet flexible film on the hair, and thus, the compositions of the present invention can be developed as 100% natural hair fixative compositions. Moreover, pullulan can be used in the present invention as a hair fixative without the use of lacquers (e.g., natural products such as shellac).

Pullulan, while being completely water soluble, is said to be insoluble in most organic compounds. Surprisingly, stable pullulan formulations have been made containing various water/alcohol solvent combinations, comprising from about 15 to 35 percent monohydric alcohol and water in an amount that is greater than about 50 percent, which not only minimize the aforementioned negative effects; but, also improve the drying time of the hair fixative composition as the amount of monohydric alcohol is increased. Therefore, the beneficial reduction in drying time is surprising because it is observed with an increase in the amount of monohydric alcohol and the compositions are stable. As shown in Example III below, drying times decrease when the amounts of ethanol are increased in compositions containing pullulan. The drying time is enhanced within a range of about 10 to 40 percent based on the measurable parameter, percent weight loss. Further, the hair fixative compositions of the present invention are stable as no precipitate of pullulan is found after at least two months.

To prepare the compositions of the present invention, pullulan, in powder form, is dissolved in water. Pullulan is available in powder form from the Hayashibara Biochemical Laboratories, Inc., Okayama, Japan. Other forms of pullulan can be used in the present invention as well. The pullulan used in the compositions of the present invention is free of hydrophobic moieties, and has a molecular weight of about 5,000 to 5,000,000, and preferably, about 275,000 to 1,000,000. Pullulan is an alpha-glucan containing mainly maltotriose as repeating units linearly joined through α-(1,6) glycosidic linkages. Pullulan is not branched and as used in the present invention can include derivatives such as trimethylsiloxysilylcarbamoyl pullulan, cholesterol/HDI/pullulan copolymer, cholesteryl hexyl dicarbamate pullulan. The amount of pullulan used in the present invention is about 0.5 to 15 percent, more preferably 5 to 10 percent by weight of the composition. Although the final composition comprises the hydroalcoholic solvent system, pullulan is prepared initially in an aqueous solution. Any optional ingredients or additional hair fixative components are added to the aqueous pullulan solution and mixed for about at least 30 minutes. The monohydric alcohol is added as the final ingredient. The monohydric alcohol is one that has a boiling point of less than 100° C. The alcohol preferably has 1 to 4 carbon atoms, and includes, for example, methanol, ethanol, propanol, isopropanol, 2-butanol or the like. More preferably, the monohydric alcohol is ethanol.

After preparing the hair fixative compositions, it can be sprayed into hands and subsequently applied to hair, or directly on to the hair. The hair is combed to ensure even coverage and can be dried and styled as usual. The application of the present hair fixative compositions sets the hair into a particular fashion and holds the hairstyle. The hair fixative compositions of the present invention can be removed by rinsing with water alone, or with soap or shampoo.

A wetting agent can be added as an optional ingredient in the hydroalcoholic pullulan compositions of the present invention. The wetting agent (or emulsifier) can be polysorbate 80 or any type of anionic, cationic, amphoteric or nonionic surface active compound, particularly fatty alcohol sulfates fatty alcohol ether sulfates, alkyl-sulfonates, alkylbenzene sulfates, quaternary ammonium salts, alkyl betaine, ethoxylated alkylphenols, fatty acid alkanol amides or ethoxylated fatty acid esters. It is known to add surfactants/emulsifiers to pullulan containing compositions for stabilizing the composition and improving the spreadability of the composition onto the skin as disclosed in US2003/008221 A1. However, it has not heretofore been known to add the wetting agent to the pullulan and hydroalcoholic solvent system of the present invention having improved drying times as a hair fixative composition. The wetting agent is present in an amount of about 0.5 to 5.0 percent by weight of the composition.

Although pullulan alone provides sufficient hair fixative properties, it may be used in combination with other additional hair fixatives, including but not limited to, other polysaccharides, shellac, PVP, PVP/vinyl acetate copolymer, PVP/acrylates copolymer, PVP/DMAPA acrylates copolymer, PVP/vinylcaprolactam/DMAPA acrylates copolymer, PVP/vinylcaprolactam/DMAPA acrylates copolymer, VA/butyl maleate/isobornyl acrylate copolymer, VA/crotonates copolymer, acrylate/acrylamide copolymer, VA/crotonates/vinyl propionate copolymer, and polymeric cellulosics. Other ingredients may include viscosity controlling agents, humectants, occlusives, sunscreens and UV absorbers, emollients, antistatic agents, fragrances, and the like. Other additives include silicones and their derivatives, moisturizers, lubricants, and penetrants such as lanolin compounds, fragrances and perfumes, antioxidants, detackifying agents, combing aids and conditioning agents, neutralizers, colorants, luster modifiers, anti-dandruff agents, plasticizers, anti-microbials, glossifiers, preservatives, proteins and their derivatives, amino acids, vitamins, emulsifiers, surfactants, thickeners and rheology modifiers, gelling agents, opacifiers, stabilizers, sequestering agents, chelating agents, aesthetic enhancers, fatty acids, fatty alcohols and triglycerides, botanical extracts, plant derived actives, antioxidants, clarifying agents, and any other additive that is commonly used in a hair care or cosmetic composition.

Furthermore, pullulan is also compatible with cationic conditioning agents such as cetrimonium chloride, polyquaternium 10, fatty amines, ethoxylated fatty amines, where the conventional synthetic fixatives are not. When these same conditioning agents are used with conventional fixatives, precipitates are formed.

After preparing the hair fixative compositions, it can be sprayed into hands and subsequently applied to hair, or directly on to the hair. The hair is combed to ensure even coverage and can be dried and styled as usual. The styled hair can be held with the hair fixative compositions of the present invention for a period of time between about 5 to 8 hours.

The invention is further illustrated by the following non-limiting examples.

### EXAMPLE 1

Setting Lotion

| Ingredient | Percent |
|---|---|
| Pullulan | 6 |
| Water | 60 |
| Ethanol | 34 |

Prepare aqueous solution of pullulan and water by dissolving pullulan in water. Add ethanol.
Resulting setting lotion dries fast, is transparent and stable, feels natural and sprays well.

### EXAMPLE 2

Setting Lotion

| Ingredient | Percent |
|---|---|
| Pullulan | 7.9 |
| Water | 77 |
| Cetrimonium chloride | 0.1 |
| Ethanol | 15 |

Prepare aqueous solution of pullulan and water by dissolving pullulan in water. Add ethanol.
Resulting setting lotion dries fast, is transparent and stable, feels natural and sprays well.

### EXAMPLE 3

Comparative Study

To demonstrate that the compositions of the present invention containing high levels of monohydric alcohol and pullulan (e.g., 25 and 30 percent ethanol) exhibits improved drying times with an increase in the amount of monohydric alcohol. The comparison is made with compositions containing 0, 5, and 10 percent monohydric alcohol. Samples of each level of ethanol (i.e., 0, 5, 10, 25, and 30 percent) are prepared for 1, 5, and 10 percent pullulan. Thus, a total of 15 samples are prepared. An aqueous solution of pullulan is made by dissolving the appropriate amount of pullulan in water. Subsequently, the appropriate amount of ethanol is added. For each sample the percent weight loss of a 1 g sample at 40° C. is measured every 10 minutes.

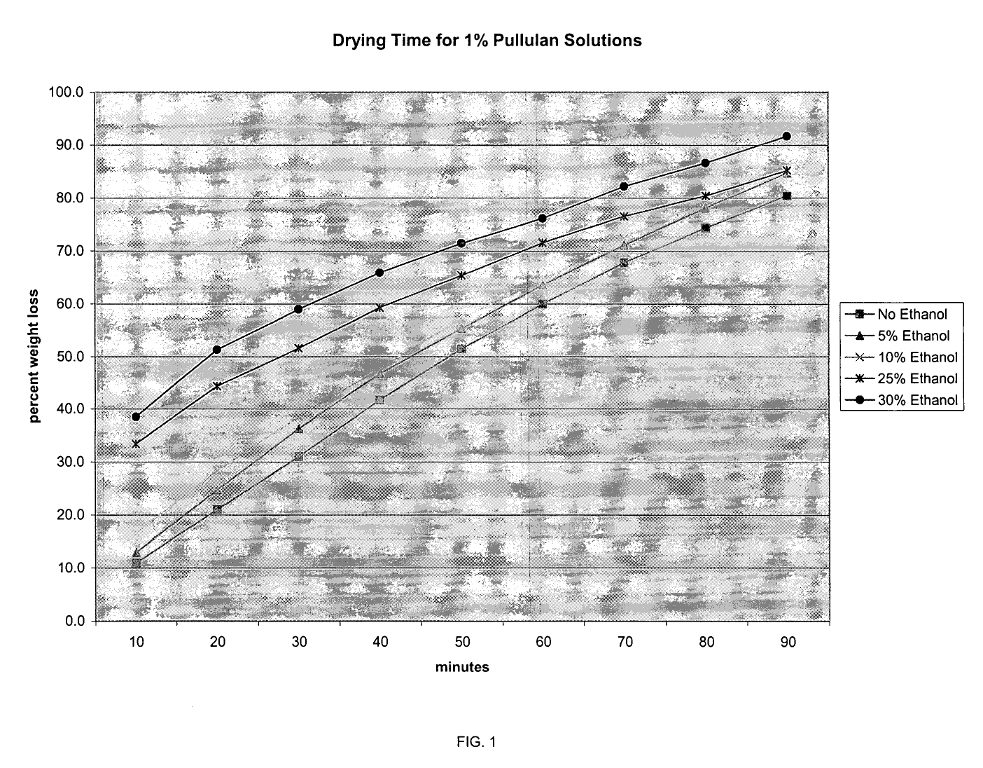

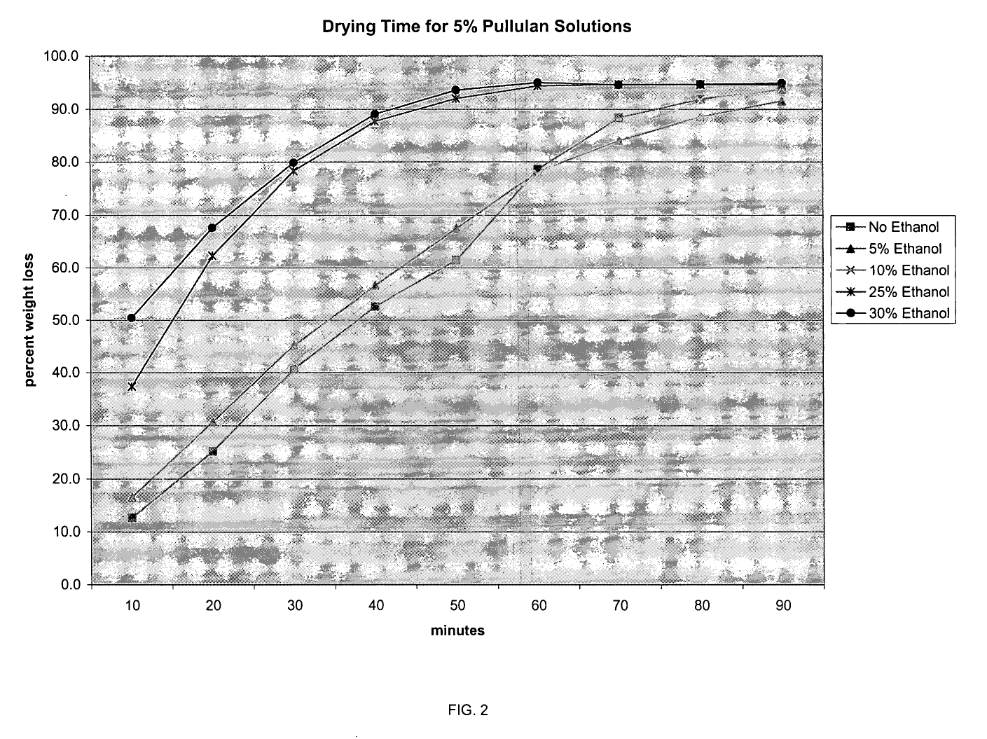

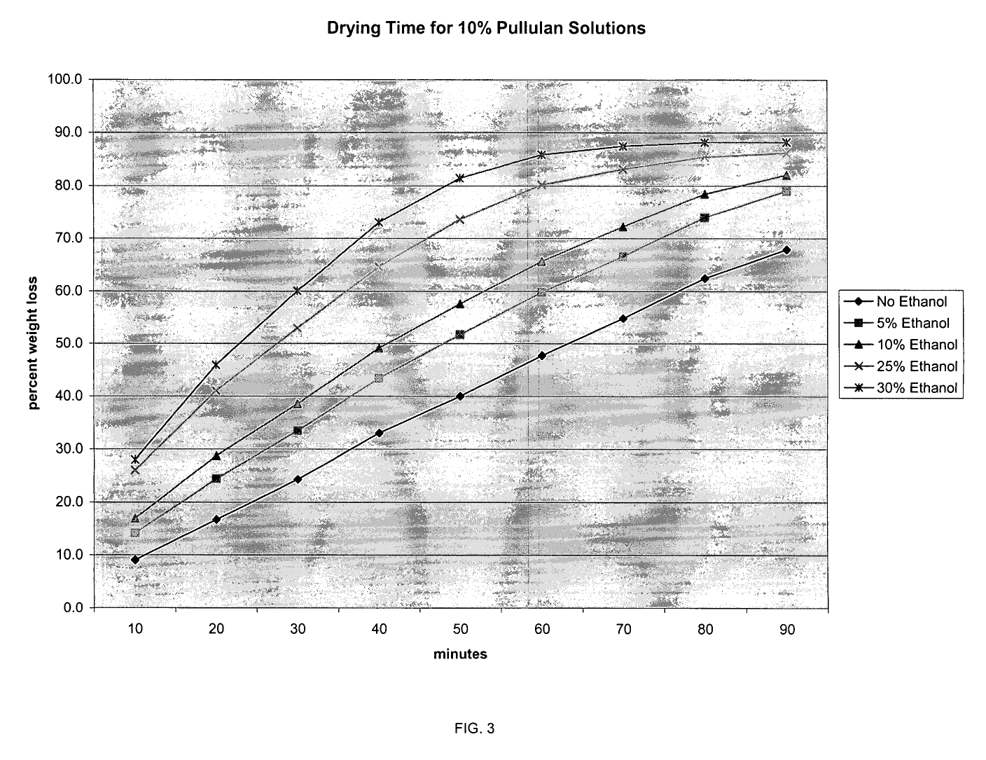

The resulting data is shown in FIGS. 1, 2, and 3. The percent weight loss for each sample indicates a decrease in drying time as percent weight loss is inversely proportional to drying time. Therefore, as percent weight loss increases, drying time decreases. It can be seen that the profile for samples containing 25 and 30 percent ethanol demonstrate a shorter drying time than the profile for samples containing 0, 5 and 10 percent ethanol which have longer drying times. The line curve for 25 and 30 percent ethanol is clustered together separate from the clustered curves for 0, 5, and 10 percent ethanol. The range of improved drying time is about 10 to 40 percent based on percent weight loss measurements between 10 and 40 minutes. The same phenomenon of clustered curves for 25 and 30 percent ethanol is seen for all 3 percentages of pullulan, namely 1, 5, and 10 percent, indicating that the samples containing the higher levels of monohydric alcohol exhibit improved drying times over lower levels of monohydric alcohol. Further, the compositions containing higher levels of ethanol, namely 25 and 30 percent, are stable, based on long-term (eight week) accelerated stability storage studies (45° C.), as no precipitate of pullulan is found.

What we claim is:

1. A hair composition having enhanced drying time comprising a linear, unbranched polysaccharide free of side chains present in an amount of about 0.5 to 15 percent by weight of the composition in a hydroalcoholic solvent comprising about 15 to 35 percent of monohydric alcohol by weight of the composition.

2. The composition of claim 1 wherein said polysaccharide is pullulan having a molecular weight of 5,000 to 5,000,000 and free of hydrophobic moieties.

3. The composition of claim 1 wherein said pullulan has a molecular weight of 275,000 to 1,000,000.

4. The composition of claim 1 wherein said monohydric alcohol has a boiling point of less than 100° C.

5. The composition of claim 4 wherein said monohydric alcohol is ethanol.

6. The composition of claim 1 further comprising a wetting agent.

7. The composition of claim 6 wherein said wetting agent is polysorbate 80.

8. The composition of claim 1 further comprising a cationic conditioning agent.

9. The composition of claim 8 wherein said conditioning agent is cetrimonium chloride.

10. A method for preparing the composition of claim 1, comprising dissolving the pullulan in water, adding additional hair fixative components, mixing the pullulan solution, and adding monohydric alcohol.

11. A process of setting the hair comprising applying the composition of claim 1 to the hair.

12. A method for increasing the drying time of a hair fixative composition comprising preparing the composition of claim 1 and applying the composition to the hair.
